(19) United States
(12) Patent Application Publication
(10) Pub. No.: US 2025/0107713 A1
(43) Pub. Date: Apr. 3, 2025

(54) Systems and Methods for Confirming Iboga Alkaloid Treatment

(71) Applicant: The Board of Trustees of the Leland Stanford Junior University, Stanford, CA (US)

(72) Inventors: Ian Kratter, Palo Alto, CA (US); Nolan R. Williams, Incline Village, NV (US); Azeezat Azeez, Menlo Park, CA (US); Malvika Sridhar, Stanford, CA (US); Manish Saggar, Palo Alto, CA (US)

(73) Assignee: The Board of Trustees of the Leland Stanford Junior University, Stanford, CA (US)

(21) Appl. No.: 18/901,503

(22) Filed: Sep. 30, 2024

Related U.S. Application Data
(60) Provisional application No. 63/586,991, filed on Sep. 29, 2023.

(51) Int. Cl. A61B 5/00; A61B 5/026; A61B 5/055

(52) U.S. Cl. CPC A61B 5/0042; A61B 5/0263; A61B 5/055; A61B 5/4848; A61B 2576/026

(57) ABSTRACT

Systems and methods for treatment verification and re-treatment with iboga alkaloids in accordance with embodiments of the invention are illustrated. One embodiment includes obtaining a first arterial spin labeling (ASL) scan of a patient's brain pre-treatment with an iboga-alkaloid for a neuropsychiatric condition, obtaining a second ASL scan of the patient's brain post-treatment, measuring changes in regional cerebral blood flow (rCBF) in a left orbitofrontal cortex, left insula, right cingulate cortex, and left putamen of the patient's brain based on the first ASL scan and the second ASL scan, determining that the treatment succeeded when either (i) rCBF increased by at least 0.1 in the left orbitofrontal cortex, or (ii) rCBF increased by any amount in the left orbitofrontal cortex and in at least two of the left insula, right cingulate cortex, and left putamen, and determining that the treatment failed when it is not determined that the treatment succeeded.

### CROSS-REFERENCE TO RELATED APPLICATIONS

The current application claims the benefit of and priority under 35 U.S.C. § 119(e) to U.S. Provisional Patent Application No. 63/586,991 entitled “Exploring the Neural Correlates of Ibogaine in Special Forces Combat Veterans through Multimodal Imaging” filed Sep. 29, 2023. The disclosure of U.S. Provisional Patent Application No. 63/586,991 is hereby incorporated by reference in its entirety for all purposes.

### FIELD OF THE INVENTION

The present invention generally relates to measuring insular blood flow to direct and verify treatment of neuropsychiatric disorders with ibogaine, ibogaine analogues, and/or pharmaceutically active salts thereof.

### BACKGROUND

Traumatic brain injury (TBI) is a major public health concern, particularly among combat veterans, contributing to a sequelae of psychiatric disorders such as post-traumatic stress disorder (PTSD), major depressive disorder (MDD), and generalized anxiety disorder (GAD). The current standard of care for TBI accompanied by PTSD includes rehabilitation therapy, talk therapy, and medication.

Functional magnetic resonance imaging (fMRI) is a class of medical imaging techniques that enable non-invasive measurement of neuronal activity in the brain of a live, awake, subject. Arterial spin labeling (ASL) and blood oxygen level dependent (BOLD) imaging are two different types of fMRI that both can be used to estimate cerebral blood flow (CBF) but have different operating principals. ASL uses a radio frequency pulse to magnetically label arterial blood water as an endogenous tracer. In contrast, BOLD measures responses from deoxygenated hemoglobin which is paramagnetic compared to oxygenated hemoglobin which is not. While the BOLD contrast primarily detects changes in T2 that indirectly reflect changes in CBF, ASL perfusion contrast is essentially based on alternations in T1 induced directly by changes in regional blood flow.

Iboga alkaloids are alkaloid constituents of the Tabernathe iboga. Ibogaine, ibogaline, ibogamine and tabernathine are representative molecules, and are psychoactive. Despite being known for over a century, the full scope of effects on the human body have remained unclear. For example, ibogaine has been reported to be effective in treating addition to opioid and stimulant drugs, but has remained a less used pharmaceutical option due to its significant hallucinogenic, neurotoxic, and cardiovascular side effects. Approximately 1 in 300 people may suffer from cardiac arrest when treated with ibogaine. Given the dangers of the drug, ibogaine has been classified in the United States as a Schedule I controlled substance. Compositions and methods are described herein for administration of iboga alkaloids with cardiovascular side effects, such as ibogaine.

The foregoing examples of the related art and limitations related therewith are intended to be illustrative and not exclusive. Other limitations of the related art will become apparent to those of skill in the art upon a reading of the specification and a study of the drawings.

### SUMMARY OF THE INVENTION

Systems and methods for treatment verification and re-treatment with iboga alkaloids in accordance with embodiments of the invention are illustrated. One embodiment includes a method for verifying treatment with iboga alkaloids, including obtaining a first arterial spin labeling (ASL) scan of a patient's brain pre-treatment with an iboga-alkaloid for a neuropsychiatric condition, obtaining a second ASL scan of the patient's brain post-treatment, measuring changes in regional cerebral blood flow (rCBF) in a left orbitofrontal cortex, a left insula, a right cingulate cortex, and a left putamen of the patient's brain based on the first ASL scan and the second ASL scan, determining that the treatment succeeded when either (i) rCBF increased by at least 0.1 in the left orbitofrontal cortex, or (ii) rCBF increased by any amount in the left orbitofrontal cortex and in at least two of the left insula, right cingulate cortex, and left putamen, and determining that the treatment failed when it is not determined that the treatment succeeded.

In a further embodiment, the method further includes steps for re-treating the patient upon a determination the treatment failed and that rCBF increased less than 0.1 in the left orbitofrontal cortex.

In still another embodiment, the method further includes steps for re-treating the patient upon a determination the treatment failed and that rCBF increased in exactly one of the left insula, right cingulate cortex, and left putamen.

In a still further embodiment, the method further includes steps for re-treating the patient upon a determination the treatment failed, that rCBF increased less than 0.1 in the left orbitofrontal cortex, and that rCBF increased in exactly one of the left insula, right cingulate cortex, and left putamen.

In yet another embodiment, the neuropsychiatric condition is traumatic brain injury.

In a yet further embodiment, the neuropsychiatric condition is post-traumatic stress disorder.

In another additional embodiment, the neuropsychiatric condition is major depressive disorder.

In a further additional embodiment, the second ASL scan of the patient's brain is obtained between 2- and 5-days post treatment.

One embodiment includes a system for verifying treatment with iboga alkaloids, including an MRI machine capable of ASL imaging, and a treatment verification device, including a processor, and a memory, the memory storing a treatment verification application that configures the processor to obtain, from the MRI machine, a first ASL scan of a patient's brain pre-treatment with an iboga-alkaloid for a neuropsychiatric condition, obtain, from the MRI machine, a second ASL scan of the patient's brain post-treatment, measure changes in rCBF in a left orbitofrontal cortex, a left insula, a right cingulate cortex, and a left putamen of the patient's brain based on the first ASL scan and the second ASL scan, provide a determination that the treatment succeeded when either (i) rCBF increased by at least 0.1 in the left orbitofrontal cortex, or (ii) rCBF increased by any amount in the left orbitofrontal cortex and in at least two of the left insula, right cingulate cortex, and left putamen, and provide a determination that the treatment failed when it is not determined that the treatment succeeded.

In another embodiment again, the treatment verification application further configures the processor to provide a recommendation to re-treat the patient upon a determination the treatment failed and that rCBF increased less than 0.1 in the left orbitofrontal cortex.

In a further embodiment again, the treatment verification application further configures the processor to provide a recommendation to re-treat the patient upon a determination the treatment failed and that rCBF increased in exactly one of the left insula, right cingulate cortex, and left putamen.

In still yet another embodiment, the treatment verification application further configures the processor to provide a recommendation to re-treat the patient upon a determination the treatment failed, that rCBF increased less than 0.1 in the left orbitofrontal cortex, and that rCBF increased in exactly one of the left insula, right cingulate cortex, and left putamen.

In a still yet further embodiment, the neuropsychiatric condition is traumatic brain injury.

In still another additional embodiment, the neuropsychiatric condition is post-traumatic stress disorder.

In a still further additional embodiment, the neuropsychiatric condition is major depressive disorder.

In still another embodiment again, the second ASL scan of the patient's brain is obtained between 2- and 5-days post treatment.

One embodiment includes a non-transitory computer-readable medium storing instructions that when executed by a processor configure the processor to perform the steps of obtaining a ASL scan of a patient's brain pre-treatment with an iboga-alkaloid for a neuropsychiatric condition, obtaining a second ASL scan of the patient's brain post-treatment, measuring changes in rCBF in a left orbitofrontal cortex, a left insula, a right cingulate cortex, and a left putamen of the patient's brain based on the first ASL scan and the second ASL scan, determining that the treatment succeeded when either (i) rCBF increased by at least 0.1 in the left orbitofrontal cortex, or (ii) rCBF increased by any amount in the left orbitofrontal cortex and in at least two of the left insula, right cingulate cortex, and left putamen, and determining that the treatment failed when it is not determined that the treatment succeeded.

In a still further embodiment again, the stored instructions when executed by the processor further configure the processor to perform the steps of providing a recommendation of re-treating the patient upon a determination the treatment failed and that rCBF increased less than 0.1 in the left orbitofrontal cortex.

In yet another additional embodiment, the stored instructions when executed by the processor further configure the processor to perform the steps of providing a recommendation of re-treating the patient upon a determination the treatment failed and that rCBF increased in exactly one of the left insula, right cingulate cortex, and left putamen.

In a yet further additional embodiment, the stored instructions when executed by the processor further configure the processor to perform the steps of providing a recommendation of re-treating the patient upon a determination the treatment failed, that rCBF increased less than 0.1 in the left orbitofrontal cortex, and that rCBF increased in exactly one of the left insula, right cingulate cortex, and left putamen.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a system for verifying ibogaine treatment in accordance with an embodiment of the invention.

FIG. 2 illustrates a verification device in accordance with an embodiment of the invention.

FIG. 3 is a flow chart for a treatment verification process in accordance with an embodiment of the invention.

FIG. 4 is a set of charts displaying change in rCBF at the left insula cluster associated with improvement in symptoms in accordance with an embodiment of the invention.

FIG. 5 is a chart demonstrating improvement in symptoms associated with increase in rCBF at the left cingulate cluster in accordance with an embodiment of the invention.

FIG. 6 is a table showing the effect sizes of rCBF changes across time points, from baseline to immediate post treatment, and baseline to 1-month post treatment, for different brain regions in accordance with an embodiment of the invention.

FIG. 7 is a table showing descriptive statistics for each brain sub-cluster at different time points in accordance with an embodiment of the invention.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the composition and all measurements made are at about 25° C., unless otherwise specified.

“Administration” refers to introducing an agent, such as an iboga alkaloid, into a subject or patient. Typically, an effective amount is administered, which amount can be determined by the treating physician or the like. Any route of administration, such as oral, topical, subcutaneous, peritoneal, intra-arterial, inhalation, vaginal, rectal, nasal, introduction into the cerebrospinal fluid, or instillation into body compartments can be used. The agent, such as an iboga alkaloid, may be administered by direct blood stream delivery, e.g. sublingual, buccal, intranasal, or intrapulmonary administration.

The related terms and phrases “administering” and “administration of’, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Ibogaine” refers to the compound:

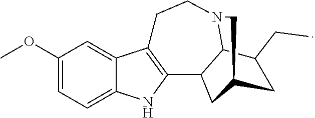

as well as pharmaceutically acceptable salts and pharmaceutically acceptable solvates thereof. It should be understood that where “ibogaine” is mentioned herein, one or more polymorphs of ibogaine can be utilized and are contemplated. Ibogaine is isolated from Tabernanth iboga, a shrub of West Africa. Reference to a derivative of ibogaine or an ibogaine derivative intends a compound other than ibogaine but based on its molecular core, and non-limiting examples of ibogaine derivatives are provided, for example, in WO2017/184531

“Noribogaine” refers to the compound:

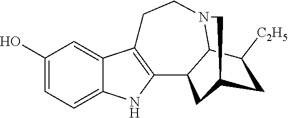

as well as pharmaceutically acceptable salts thereof or solvates thereof. Noribogaine can be prepared by demethylation of naturally occurring ibogaine. Demethylation may be accomplished by conventional techniques such as by reaction with boron tribromide/methylene chloride at room temperature followed by conventional purification. See, for example, Huffman, et al., J. Org. Chem 50:1460 (1985). Noribogaine can be synthesized as described, for example, in U.S. Patent Pub. Nos. 2013/0165647, 2013/0303756, and 2012/0253037, PCT Patent Publication No. WO 2013/040471 (includes description of making noribogaine polymorphs), and U.S. Pat. No. 9,617,274, each of which is incorporated herein by reference in its entirety.

“Neuropsychiatric” is used herein with reference to disorders of affect, cognition, and/or behavior that arise from a disorder in cerebral function and/or from indirect effects of an extracerebral disease. Neuropsychiatric disorders include neurological disorders and neurodegenerative disorders.

As used herein, the term “patient” or “subject” refers to mammals and includes humans and non-human mammals.

The phrase “pharmaceutically acceptable” is employed herein to refer to those compounds, salts, compositions, dosage forms, etc., which are within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

“Pharmaceutically acceptable salt” refers to salts, including pharmaceutically acceptable partial salts, of a compound, which salts are derived from a variety of organic and inorganic counter ions well known in the art and include, by way of example only, hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, oxalic acid and the like, and when the molecule contains an acidic functionality, include, by way of example only, sodium, potassium, calcium, magnesium, ammonium, tetraalkylammonium, and the like.

“Therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, in the context of treating a neurodegenerative disease or a movement disorder and/or symptoms thereof, refers to an amount of the agent that attenuates the disease or disorder; attenuates, reverses, or reduces the severity of a symptom or symptoms thereof; and/or prevents, delays, or reduces the severity of progression of the disease or disorder.

A “therapeutic level” of a drug is an amount of iboga alkaloid or pharmaceutically acceptable salt or solvate thereof that is sufficient to treat or prevent the disease or disorder and/or symptoms thereof, but not high enough to pose any significant risk to the patient. Therapeutic levels of drugs can be determined by tests that measure the actual concentration of the compound in the blood of the patient. This concentration is referred to as the “serum concentration.”

The term “treating” is used herein, for instance, in reference to methods of treating a neuropsychiatric disorder, and generally includes the administration of a compound or composition which reduces the frequency of, or delays the onset of, symptoms of a medical condition (e.g., a neuropsychiatric disorder or a neurological disorder or a neurodegenerative disorder) in a subject relative to a subject not receiving the compound or composition. This can include reversing, reducing, or arresting the symptoms, clinical signs, and underlying pathology of a condition in a manner to improve or stabilize a subject's condition.

The term “unit dose” refers to a dose of drug provided to a patient to provide a therapeutic result, independent of the weight of the patient. The unit dose can be a standard form (e.g., a tablet or capsule). The unit dose may be administered as a single dose or a series of subdoses that collectively equal the single dose.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

The terms “tangible” and “non-transitory,” as used herein, are intended to describe a computer-readable storage medium (or “memory”) excluding propagating electromagnetic signals, but are not intended to otherwise limit the type of physical computer-readable storage device that is encompassed by the phrase computer-readable medium or memory. For instance, the terms “non-transitory computer-readable medium” or “tangible memory” are intended to encompass types of storage devices that do not necessarily store information permanently, including for example, random access memory (RAM). Program instructions and data stored on a tangible computer-accessible storage medium in non-transitory form may further be transmitted by transmission media or signals such as electrical, electromagnetic, or digital signals, which may be conveyed via a communication medium such as a network and/or a wireless link.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Objective Treatment Verification

Treatment with iboga alkaloids have been demonstrated to have therapeutic effect for a number of neuropsychiatric conditions including (but not limited to) post traumatic stress disorder (PTSD), major depressive disorder (MDD), traumatic brain injury (TBI), and anxiety. Treatment with iboga alkaloids is described in U.S. patent application Ser. No. 18/467,324, titled “Compositions of Iboga Alkaloids and Methods of Treatment”, filed Sep. 14, 2023, and in U.S. patent application Ser. No. 18/467,343, titled “Methods of Treatment With an Iboga Alkaloid”, filed Sep. 14, 2023. The disclosures of U.S. patent application Ser. Nos. 18/467,234 and 18/467,343 are incorporated by reference in their entirety.

While therapeutic effect is often seen by clinicians and reported by patients post treatment, an objective measurement of treatment verification is desirable. Patients may be grateful to have a non-biased confirmation that treatment has worked given the psychiatric effects of their condition. Similarly, medical practitioners may benefit from objective reporting of patient condition. Systems and methods described herein provide such objective confirmation that treatment has been successful by measuring cerebral blood flow in specific regions of the brain. In many embodiments, regional cerebral blood flow (rCBF) is measured at the left orbitofrontal cortex, the left insula, the right cingulate cortex, and the left putamen. In numerous embodiments, arterial spin labeling (ASL) scan is used to measure rCBF rather than blood-oxygen level dependent (BOLD) imaging. For the instant use case, ASL provides more reproducible measurement than BOLD. A pre-treatment and post-treatment scan can be taken, and the change in rCBF in the particular brain regions is measured. In many embodiments, the post-treatment scan is taken between 2- and 5-days post treatment. However, the second scan may be taken at least 1 month post treatment.

In many embodiments, if the change in rCGF from pre-treatment to post-treatment on a normalized individual level (z-score) does not meet either of the following conditions: increased by at least 0.1 in the left orbitofrontal cortex; or (ii) increased by any amount in the left orbitofrontal cortex and in at least two of the left insula, right cingulate cortex, and left putamen; then the treatment is considered a failure. In numerous embodiments, a determination that any increase in z-score, but not by at least 0.1, indicates that retreatment should occur. Based on currently available data, 0.1 represents a 33^{rd} percentile change at the group level. As additional data is acquired, the 0.1 threshold may change. As can be readily appreciated, the threshold value can be determined based on the particular patient population without departing from the scope or spirit of the invention.

### II.A. Treatment Verification Systems

Treatment verification systems in accordance with embodiments of the invention are discussed herein. In a variety of embodiments, treatment verification systems include a magnetic resonance imaging (MRI) machine. MRI machines as contemplated herein can perform ASL scans of patient brains. A treatment verification device is used to obtain pre-treatment and post-treatment ASL scans produced by the MRI machine, and measure the change in rCBF in the left orbitofrontal cortex, the left insula, the right cingulate cortex, and the left putamen of the patient's brain.

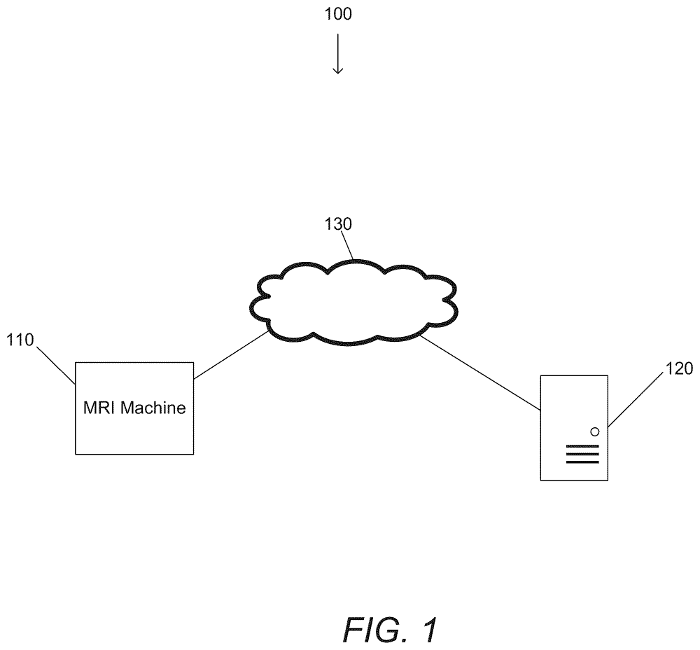

Turning now to FIG. 1, a treatment verification system in accordance with an embodiment of the invention is illustrated. Treatment verification system 100 includes an MRI machine 110 and treatment verification device 120. Treatment verification device 120 obtains ASL scans from the MRI machine 110 via a network 130. In numerous embodiments, the network is a hospital intranet. However, the network can be the Internet, an intranet, or any other network or combination of networks. In various embodiments, scans are manually transferred between computing devices in the system, for example via a compact disc or flash drive.

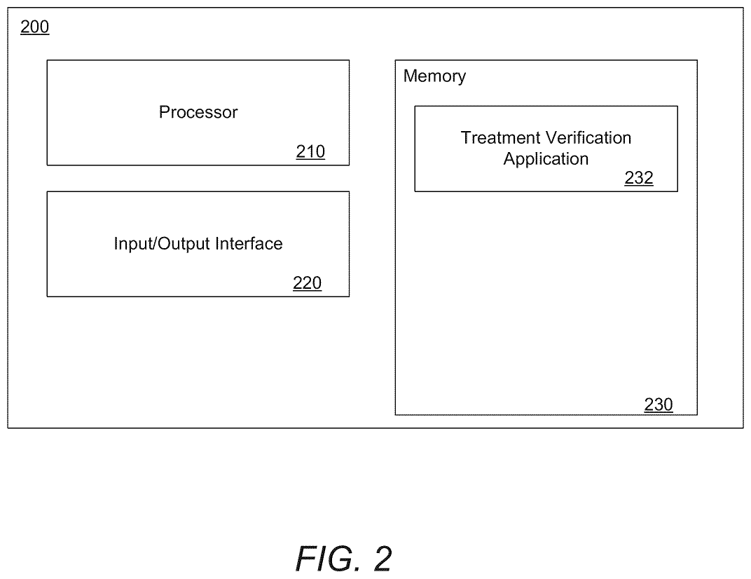

A block diagram for a treatment verification device in accordance with an embodiment is illustrate in FIG. 2. Treatment verification device 200 includes a processor 210. Processors can be any logic processing circuitries or combination of circuitries including (but not limited to) central processing units (CPUs), graphics processing units (GPUs), field-programmable gate arrays (FPGAs), application-specific integrated circuits (ASICs), and/or any other circuitry as appropriate to the requirements of specific applications of embodiments of the invention. Treatment verification device further includes an input/output (I/O) interface 220. I/O interfaces are capable of receiving AFI scans. In some embodiments, I/O interfaces are capable of interfacing with networks.

Treatment verification device 200 further includes a memory 230. Memory can be volatile memory, non-volatile memory, or a combination thereof. The memory 230 stores a treatment verification application (232) which includes machine readable instructions that configure the processor to carry out various treatment verification processes as described herein. While a particular architecture for a treatment verification device is illustrated in FIG. 2, as can be readily appreciated, any number of different computing platform architectures cam be used as appropriate to the requirements of specific applications of embodiments of the invention. Treatment verification processes are discussed further below.

### II.B. Treatment Verification Processes

Verification of treatment with ibogaine involves taking a pre-treatment, or “baseline” ASL scan of the patient's brain. Similar ASL scans of the patient's brain post-treatment can be used to verify efficacy of treatment by measuring the change in rCBF in particular areas of the brain. In numerous embodiments, the ASL scans are pseudo continuous ASL scans (pcASL). As noted, above, in many embodiments, treatment is considered a success if the change in rCBF from pre-treatment to post-treatment on a normalized individual level (z-score) increased by at least 0.1 in the left orbitofrontal cortex or increased by any amount in the left orbitofrontal cortex and in at least two of the left insula, right cingulate cortex, and left putamen. In various embodiments, when a change in rCBF is seen, but does not meet the threshold, a decision can be made to re-treat the patient with ibogaine.

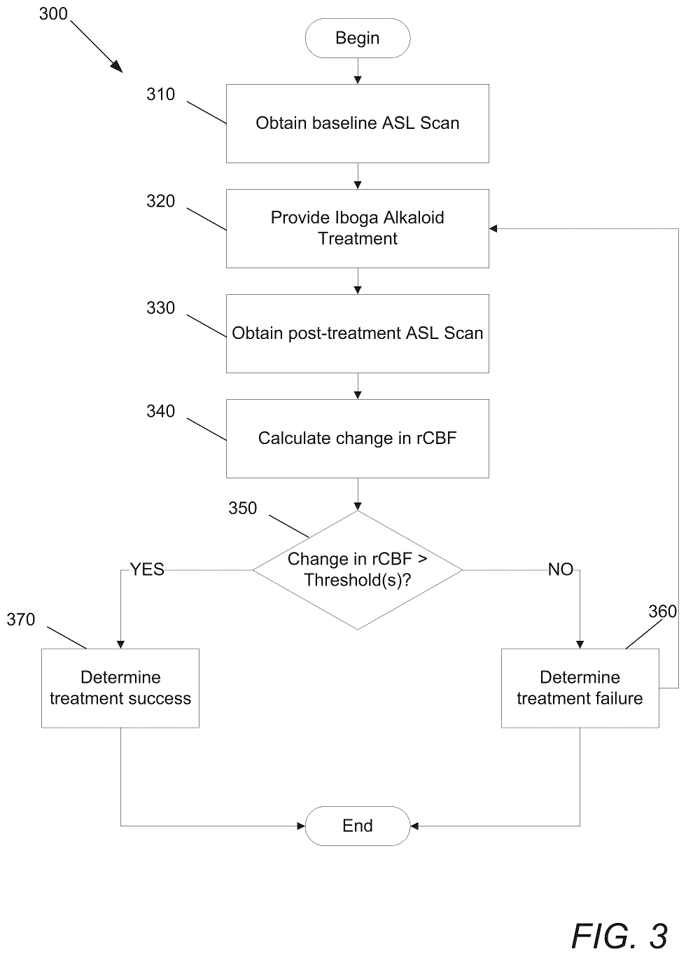

Turning now to FIG. 3, a flow chart for a treatment verification process in accordance with an embodiment of the invention is illustrated. Process 300 includes obtaining (310) a baseline ASL scan of the patient's brain. Treatment with an iboga alkaloid (320) is given to the patient. A post-treatment ASL scan is then obtained (330). In many embodiments, the post-treatment scan is obtained between 2- and 5-days post treatment. In some embodiments, the post-treatment scan is between 4- and 5 days post treatment. In various embodiments, the post-treatment scan in obtained a month post-treatment. As can be readily appreciated, the post-treatment scan may be obtained any time post treatment (e.g. 1 week, 2 weeks, 1 month, etc.) as appropriate to the requirements of specific applications of embodiments of the invention.

A change in rCBF is calculated (340) based on measurement of rCBF in each of the baseline, pre-treatment scan, and the post-treatment scan. In numerous embodiments, the rCBF is calculated for one or more of the left orbitofrontal cortex and in at least two of the left insula, right cingulate cortex, and left putamen.

In numerous embodiments, the scans are pre-processed as part of measuring rCBF in each scan. For example in many embodiments, the ASL scans are reoriented, aligned, and co-registered to an anatomical scan. In various embodiments, six motion parameters (x, y, z translations, and 3 rotations), spin labeling, and control labeling time paradigm (the zigzag pattern) are regressed from the motion time courses. This can be followed by temporal Butterworth high pass filtering (0.04-1 Hz), spatial smoothing, and normalization to a standard brain space (e.g. MNI space). The outputs can then be Z-scored and skull-stripped using a whole brain mask. Once, rCBF in the desired regions of interest are calculated, the change from pre-treatment to post-treatment for each region can simply be calculated.

Process 300 then includes determining (350) if the change in calculated rCBF has exceeded a threshold. In many embodiments, different brain regions have different thresholds. For example, in various embodiments, the threshold for change in rCBF for the left orbitoprefrontal cortex is 0.1, but the threshold for the left insula, right cingulate cortex, and left putamen is at or near 0. However, as noted above, while the threshold for the left insula, right cingulate cortex, and left putamen is at or near 0, two of the three regions must exceed their thresholds. Additionally, the threshold for a given region may be modified depending on patient population. For example, in many embodiments, the threshold is between 0.1 and 0.7 for the orbitoprefrontal cortex, but any number of different thresholds can be determined for a given region for a particular patient population.

If the thresholds are not sufficiently met, then the treatment is determined (360) to be a failure. In the event of a failure, retreatment can occur. In numerous embodiments, the decision to re-treat occurs if an increase in rCBF is seen, but not enough to exceed the thresholds. If the thresholds are sufficiently met, then the treatment is determined (370) to be a success.

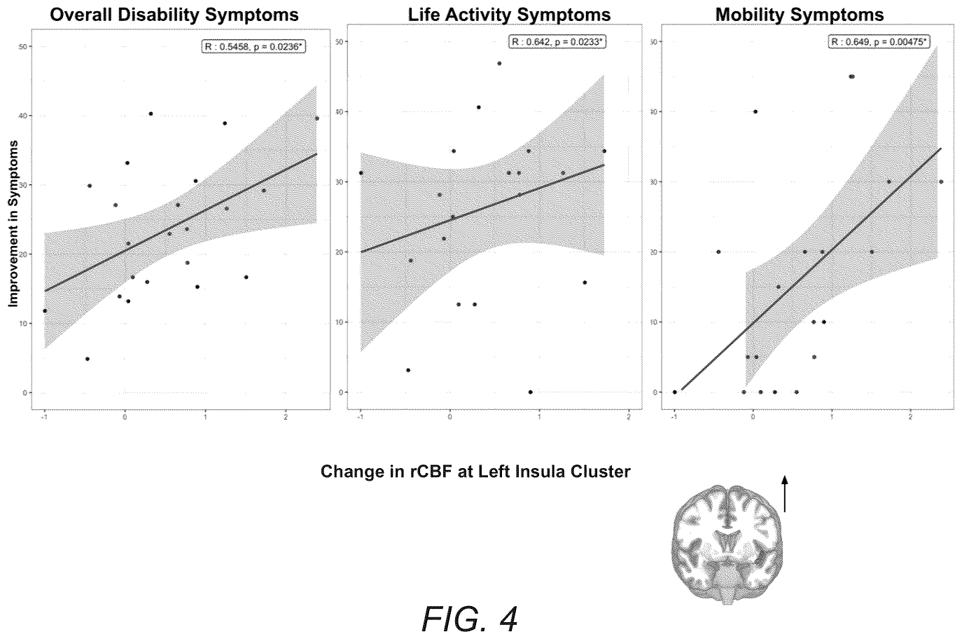

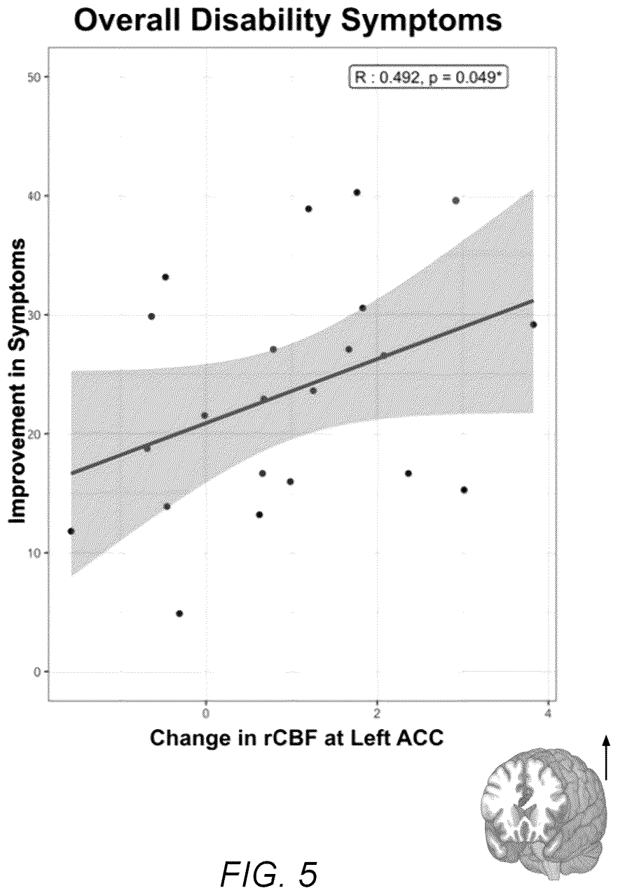

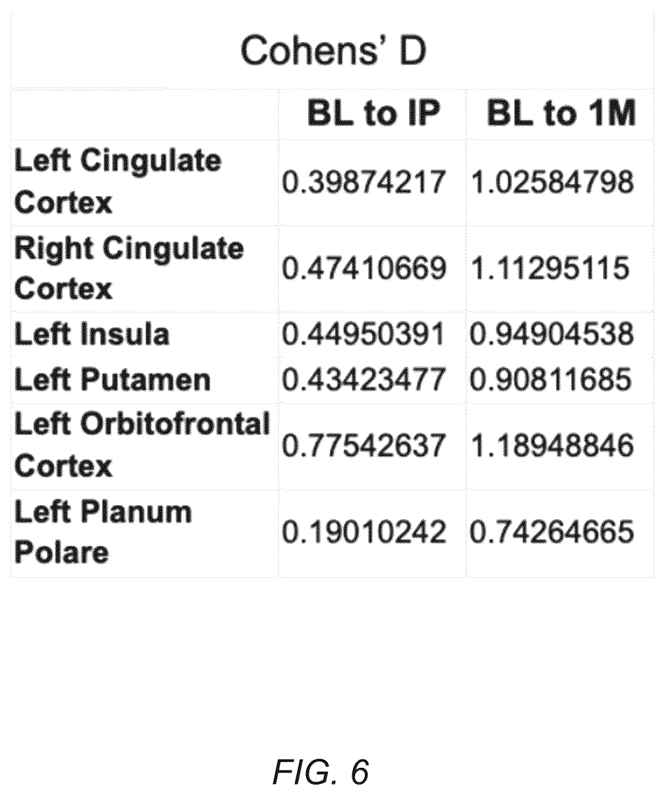

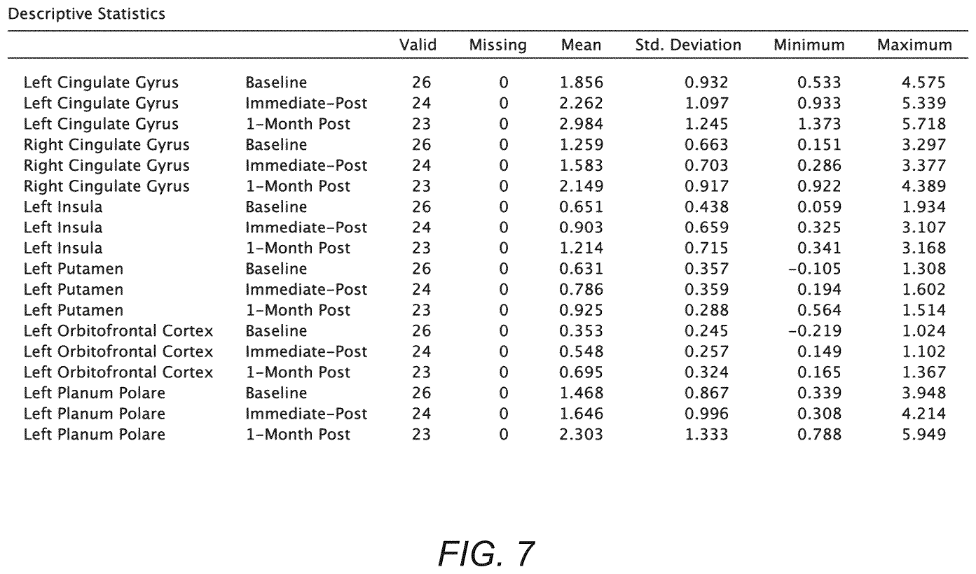

Turning now to FIG. 4, charts displaying data demonstrating change in rCBF at the left insula cluster associated with improvement in symptoms is illustrated. Change in WHODAS score vs. rCBF change in the left insula from baseline to 1-month post treatment is shown. Similarly, FIG. 5 illustrates data demonstrating improvement in symptoms associated with increase in rCBF at the left cingulate cluster, showing change in WHODAS score vs rCBF change from baseline to 1-month post treatment. FIG. 6 is a table showing the effect sizes of rCBF changes across time points, from baseline to immediate post treatment (4-5 days), and baseline to 1-month post treatment, for different brain regions. FIG. 7 is a table showing descriptive statistics for each sub-cluster at all three time points. Data for FIGS. 4-7 are calculated from the same study of U.S. military veterans.

Although specifics are discussed above, many different treatment verification methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for verifying treatment with iboga alkaloids, comprising:
obtaining a first arterial spin labeling (ASL) scan of a patient's brain pre-treatment with an iboga-alkaloid for a neuropsychiatric condition;
obtaining a second ASL scan of the patient's brain post-treatment;
measuring changes in regional cerebral blood flow (rCBF) in a left orbitofrontal cortex, a left insula, a right cingulate cortex, and a left putamen of the patient's brain based on the first ASL scan and the second ASL scan;
determining that the treatment succeeded when either:
(i) rCBF increased by at least 0.1 in the left orbitofrontal cortex; or
(ii) rCBF increased by any amount in the left orbitofrontal cortex and in at least two of the left insula, right cingulate cortex, and left putamen; and
determining that the treatment failed when it is not determined that the treatment succeeded.

2. The method of claim 1, further comprising re-treating the patient upon a determination the treatment failed and that rCBF increased less than 0.1 in the left orbitofrontal cortex.

3. The method of claim 1, further comprising re-treating the patient upon a determination the treatment failed and that rCBF increased in exactly one of the left insula, right cingulate cortex, and left putamen.

4. The method of claim 1, further comprising re-treating the patient upon a determination the treatment failed, that rCBF increased less than 0.1 in the left orbitofrontal cortex, and that rCBF increased in exactly one of the left insula, right cingulate cortex, and left putamen.

5. The method of claim 1, wherein the neuropsychiatric condition is traumatic brain injury.

6. The method of claim 1, wherein the neuropsychiatric condition is post-traumatic stress disorder.

7. The method of claim 1, wherein the neuropsychiatric condition is major depressive disorder.

8. The method of claim 1, wherein the second ASL scan of the patient's brain is obtained between 2- and 5-days post treatment.

9. A system for verifying treatment with iboga alkaloids, comprising:
a magnetic resonance imaging (MRI) machine capable of arterial spin labeling (ASL) imaging; and
a treatment verification device, comprising:
a processor; and
a memory, the memory storing a treatment verification application that configures the processor to:
obtain, from the MRI machine, a first ASL scan of a patient's brain pre-treatment with an iboga-alkaloid for a neuropsychiatric condition;
obtain, from the MRI machine, a second ASL scan of the patient's brain post-treatment;
measure changes in regional cerebral blood flow (rCBF) in a left orbitofrontal cortex, a left insula, a right cingulate cortex, and a left putamen of the patient's brain based on the first ASL scan and the second ASL scan;
provide a determination that the treatment succeeded when either:
(i) rCBF increased by at least 0.1 in the left orbitofrontal cortex; or
(ii) rCBF increased by any amount in the left orbitofrontal cortex and in at least two of the left insula, right cingulate cortex, and left putamen; and
provide a determination that the treatment failed when it is not determined that the treatment succeeded.

10. The system of claim 9, wherein the treatment verification application further configures the processor to provide a recommendation to re-treat the patient upon a determination the treatment failed and that rCBF increased less than 0.1 in the left orbitofrontal cortex.

11. The system of claim 9, wherein the treatment verification application further configures the processor to provide a recommendation to re-treat the patient upon a determination the treatment failed and that rCBF increased in exactly one of the left insula, right cingulate cortex, and left putamen.

12. The system of claim 9, wherein the treatment verification application further configures the processor to provide a recommendation to re-treat the patient upon a determination the treatment failed, that rCBF increased less than 0.1 in the left orbitofrontal cortex, and that rCBF increased in exactly one of the left insula, right cingulate cortex, and left putamen.

13. The system of claim 9, wherein the neuropsychiatric condition is traumatic brain injury.

14. The system of claim 9, wherein the neuropsychiatric condition is post-traumatic stress disorder.

15. The system of claim 9, wherein the neuropsychiatric condition is major depressive disorder.

16. The system of claim 9, wherein the second ASL scan of the patient's brain is obtained between 2- and 5-days post treatment.

17. A non-transitory computer-readable medium storing instructions that when executed by a processor configure the processor to perform the steps of:
obtaining a first arterial spin labeling (ASL) scan of a patient's brain pre-treatment with an iboga-alkaloid for a neuropsychiatric condition;
obtaining a second ASL scan of the patient's brain post-treatment;
measuring changes in regional cerebral blood flow (rCBF) in a left orbitofrontal cortex, a left insula, a right cingulate cortex, and a left putamen of the patient's brain based on the first ASL scan and the second ASL scan;
determining that the treatment succeeded when either:
(i) rCBF increased by at least 0.1 in the left orbitofrontal cortex; or
(ii) rCBF increased by any amount in the left orbitofrontal cortex and in at least two of the left insula, right cingulate cortex, and left putamen; and
determining that the treatment failed when it is not determined that the treatment succeeded.

18. The non-transitory computer-readable medium of claim 17, wherein the stored instructions when executed by the processor further configure the processor to perform the steps of providing a recommendation of re-treating the patient upon a determination the treatment failed and that rCBF increased less than 0.1 in the left orbitofrontal cortex.

19. The non-transitory computer-readable medium of claim 17, wherein the stored instructions when executed by the processor further configure the processor to perform the steps of providing a recommendation of re-treating the patient upon a determination the treatment failed and that rCBF increased in exactly one of the left insula, right cingulate cortex, and left putamen.

20. The non-transitory computer-readable medium of claim 17, wherein the stored instructions when executed by the processor further configure the processor to perform the steps of providing a recommendation of re-treating the patient upon a determination the treatment failed, that rCBF increased less than 0.1 in the left orbitofrontal cortex, and that rCBF increased in exactly one of the left insula, right cingulate cortex, and left putamen.
